(19) United States
(12) Patent Application Publication
(10) Pub. No.: US 2015/0238671 A1
(43) Pub. Date: Aug. 27, 2015

(54) BLOOD FLOW MANAGEMENT METHODS AND SYSTEMS

(71) Applicant: BRAVO BIOMED, Rockland, MA (US)

(72) Inventor: Sameh Mesallum, Boston, MA (US)

(21) Appl. No.: 14/630,249

(22) Filed: Feb. 24, 2015

Related U.S. Application Data
(60) Provisional application No. 61/943,658, filed on Feb. 24, 2014.

(51) Int. Cl. A61M 1/12; A61M 1/10; A61M 25/10

(52) U.S. Cl. CPC A61M 1/125; A61M 25/10; A61M 1/1086; A61M 1/1008; A61M 2025/1095; A61M 2205/3334; A61M 2205/3344

(57) ABSTRACT

Methods and devices are provided for managing fluid flow through a body part, such as all or portions of an organ or extremity. In general, fluid inflow and fluid outflow vessels to at least a portion of a body part can be managed such that blood flow characteristics can be changed while maintaining, reducing, or increasing pressure of the associated flow. In some embodiments, pressure can be controlled while all fluid in at least a portion of the inflow and outflow vessels flows in an opposite direction.

### RELATED APPLICATIONS

This application claims priority under 35 U.S.C. §119(e) to U.S. Provisional Application Ser. No. 61/943,658 entitled “BLOOD FLOW MANAGEMENT METHODS AND SYSTEMS,” filed Feb. 24, 2014, which application is incorporated herein by reference in its entirety.

### BACKGROUND OF THE INVENTION

Occlusion of arterial blood supply to various body parts can cause severe damage to vital structures, especially if the occlusion includes a large vessel, happened acutely or subacutely, and/or was prolonged. The main reason for organ or extremity damage is the lack of oxygen supply as well as other nutrients delivered by the arterial high pressure blood stream. With a stroke, for example, there is a rapidly developing loss of brain function due to a disturbance in the blood vessels supplying blood to the brain. Studies have shown that millions of brain cells die each minute following initial loss of blood flow to the brain.

Various techniques are known for re-perfusing the occluded arterial supply, including direct mechanical reperfusion (balloon or dilator), elimination of occlusion (embolectomy or resection/anastomosis), bypassing the occlusion (CABG), reopening the occlusion (stent), pharmacologic dissolution (TPA for fibrinolysis, Heparin, Aspirin), etc. Each of these methods has advantages and disadvantages. However, little progress has been observed where the occluded arterial supply is in a sensitive and surgically challenging location and the affected body part is irreversibly damaged in a short period, for example, with a stroke. A thrombotic or embolic stroke can range from being totally asymptomatic to death. Large strokes tend to leave severe neurological deficits in the sensory and/or motor systems.

The pharmacologic treatment for a stroke, for example, is not as successful as in the case of the cardiac muscle. The use of fibrinolytic agents, namely TPA, should be achieved within a three hour window from incidence. There is also heightened risk of cerebral arterial or parenchymal bleeding that does not exist in the case of coronary reperfusion by TPA. The location and anatomy of the cerebral blood vessels make them more challenging to mechanical reperfusion by catheters, balloons, and/or stents. Surgical trials at embolectomy are tried in the case of large occlusion of a proximal cerebral blood vessel. Results are inconsistent due to the rapidity of brain cell injury and the irreversibility of their viable functions. The only networks of capillaries vasculature supplying the neuronal structures other than the arterial network include the venous and the lymphatic networks. Blood flows from the arterial side to small arteries named arterioles to end in a fine capillary network that supplies the tissues on a cellular microscopic level. On the same level of capillaries, the venous network forms to collect venous blood and form bigger vessels named venules that eventually coalesce to form the cerebral veins. The lymphatic capillary network runs parallel to the venous system in general plus extra fluid system represented by the CSF circulation through specialized tissues surrounding the brain.

### SUMMARY OF THE INVENTION

Accordingly, there remains a need for methods and devices for temporarily or permanently restoring oxygenated blood supply to affected body parts. Methods and devices are provided for managing fluid flow to overcome or circumvent an occlusion, for example, within a body part, such as all or portions of an organ or extremity. In one embodiment, a method for managing fluid flow includes coupling a flow control system to arterial and/or venous capillaries proximate to an occlusion. According to some embodiments, the flow control system includes extra-corporeal pumps connected to catheters. The catheters can be introduced into blood vessels before, after, and at either side of an obstruction. The flow control system can be configured to manipulate blood flow characteristics surrounding the obstruction. For example, the flow control system can affect any one or more of rate of flow, pressure of flow, and direction of flow. In some examples, the flow control system can also be configured to oscillate flow in combination or separately from rate, pressure, and direction control. In some examples, the oscillation of flow can resolve obstructions. In other examples, manipulation of flow rate and pressure can be used to resolve obstructions in blood vessels. In some implementations, the flow control system can employ intra-corporeal pumps instead of and/or in conjunction with extra-corporeal pumps to manipulate flow characteristics.

In one embodiment, the flow control system can form blockages in blood vessels surrounding an occlusion. For example, balloon structures can be provided at the ends of catheters. The balloon structures can be inflated to seal a blood vessel and facilitate complete control of flow characteristics in a desired area of the patient's body. According to one embodiment, the flow control system is configured to oscillate blood flow resolving stroke/blood clot conditions in a highly sensitive portion of a patients' body. In some examples, oscillation of blood flow can include reversing blood flow (orthodromic flow) followed by restoring antidromic flow. According to some implementations, repeatedly switching between flow directions can dislodge blood clots. Further, alternating between any one or more of high, normal, and low flow rates, high, normal, and low pressure flows can also be used in conjunction with or separately from direction switching by the flow control system to resolve occlusions, clots, etc.

According to some embodiments, the flow control system can be connected to a buffer area of the patient. The buffer area can include a normal blood flow (e.g., unaffected by the occlusion). In one example, a buffer area includes the femoral arteries of the patient. The flow control system can capture blood from the femoral region and use the buffered blood flow obtained to facilitate manipulation of the blood flow characteristics in the regions of a blockage or clot.

According to one aspect, a method for managing blood flow in a body part is provided. The method comprises coupling a flow control system to the body part, the flow control system configured to provide at least one flow path; managing blood flow characteristics in at least one vessel connected to the body part along the at least one flow path; wherein coupling the flow control system to the body part includes placing a catheter and an intracorporeal pump within the catheter into the at least one vessel; and manipulating at least one of rate, volume, pressure, and direction of flow within the at least one vessel with the intracorporeal pump.

In one embodiment, the catheter and pump is placed entirely within the vessel. In one embodiment, the method further comprises expanding an expansion member on the distal end of the catheter to block unmanaged blood flow in the at least one vessel. In one example, unmanaged blood flow includes blood flow that is not controlled through the catheter. In one embodiment, the method further comprises managing blood flow from an inlet positioned on one side of the expansion member to an outlet positioned on an opposite side of the expansion member. In one embodiment, managing the blood flow includes operating the intracorporeal pump to affect at least one of rate, volume, pressure, and direction of blow flow. In one embodiment, the method further comprises switching between flow states, wherein the switching between flow states includes switching between at least one of high flow, normal flow, and low flow coupled with at least one of high pressure, normal pressure, and low pressure. In one embodiment, the flow states include flow direction.

In one embodiment, the method further comprises connecting a distal end of the catheter to a buffer zone having a normal fluid flow portion; and capturing a fluid volume from the buffer zone for delivery to the body part along the at least one flow path. In one embodiment, the method further comprises connecting the distal end of the catheter to an artery in the buffer zone; and capturing a fluid volume from the artery for delivery to the body part along the at least one flow path. In one embodiment, the method further comprises connecting a distal end of the catheter to a buffer zone having a normal fluid flow portion; and delivering a fluid volume from the buffer zone for delivery to the body part along the at least one flow path.

In one embodiment, the method further comprises connecting the distal end of the catheter to a vein in the buffer zone; and delivering a fluid volume from the body part to the vein for delivery along the at least one flow path. In one embodiment, coupling the flow control system to the body part includes placing at least a second catheter and second intracorporeal pump into another blood vessel coupled to the at least one blood vessel; and wherein manipulating the at least one of rate, volume, pressure, and direction of flow includes manipulating flow characteristics within the at least one vessel and the another blood vessel with the intracorporeal pump and the second intracorporeal pump.

According to one aspect, a system for managing blood flow in a body part is provided. The system comprises a flow control apparatus configured to provide at least one flow path to the body part; manage blood flow characteristics in at least one vessel connected to the body part along the at least one flow path; a catheter and an intracorporeal pump within the catheter configured to manipulate at least one of rate, volume, pressure, and direction of flow within the at least one vessel with the intracorporeal pump.

In one embodiment, the catheter and intracorporeal pump are configured to fit entirely within the at least one vessel. In one embodiment, the system further comprises an expansion member on the distal end of the catheter configured to block unmanaged blood flow in the at least one vessel. In one embodiment, the catheter further comprises an inlet positioned on one side of the expansion member connected to an outlet positioned on an opposite side of the expansion member. In one embodiment, the catheters is configured to manage blood flow between the inlet and outlet. In one embodiment, managing the blood flow includes operating the intracorporeal pump to affect at least one of rate, volume, pressure, and direction of blow flow.

In one embodiment, managing the blood flow comprises switching between flow states, wherein the switching between flow states includes switching between at least one of high flow, normal flow, and low flow coupled with at least one of high pressure, normal pressure, and low pressure. In one embodiment, the flow states include flow direction. In one embodiment, the catheter is connected to a buffer zone having a normal fluid flow portion; and the catheter is configured to receive a fluid volume from the buffer zone for delivery to the body part along the at least one flow path. In one embodiment, the catheter is connected to an artery in the buffer zone; and the catheter is configured to receive a fluid volume from the artery for delivery to the body part along the at least one flow path.

In one embodiment, the system further comprises connecting a distal end of the catheter to a buffer zone having a normal fluid flow portion; and delivering a fluid volume from the body part to the buffer zone along the at least one flow path. In one embodiment, the system further comprises at least a second catheter and second intracorporeal pump; wherein the flow control apparatus is configured to manipulate the at least one of rate, volume, pressure, and direction of flow within the at least one vessel and the another blood vessel with the intracorporeal pump and the second intracorporeal pump.

According to one aspect, a method for managing blood flow in a body part is provided. The method comprises coupling a flow control system to the body part, the flow control system configured to provide at least one flow path; managing blood flow characteristics in at least one vessel connected to the body part along the at least one flow path; wherein coupling the flow control system to the body part includes placing a first end of at least one catheter into the at least one vessel; connecting a second end of the at least one catheter into a buffered flow of a patient, wherein the first and second ends are fluidly connected; and manipulating at least one of rate, volume, pressure, and direction of flow within the at least one vessel through operation of a pump in fluid communication with at least one of the first or second ends.

In one embodiment, coupling the flow control system includes coupling a first end of a second catheter to another vessel fluidly connected to the at least one vessel; coupling a second end of the second catheter to a buffered flow of the patient; and manipulating a pressure associated with the manipulated flow responsive to manipulating a rate of removal of fluid relative to a rate of introduction of fluid via the at least one catheter and the second catheter.

According to one aspect, a system for managing blood flow in a body part is provided. The system comprises a flow control apparatus coupled to the body part, wherein the flow control apparatus is configured to provide at least one flow path; manage blood flow characteristics in at least one vessel connected to the body part along the at least one flow path; at least one catheter connected to the flow control apparatus, the at least one catheter having a first end of at least one catheter connected to at least one vessel; a second end of the at least one catheter connected to a buffered flow of a patient, wherein the first and second ends are fluidly connected; and a pump in fluid communication with at least one of the first or second ends configured to manipulate at least one of rate, volume, pressure, and direction of flow within the at least one vessel through operation of the pump responsive to control by the flow control apparatus.

In one embodiment, the system further comprises, wherein the flow control apparatus includes a second catheter coupled to another vessel fluidly connected to the at least one vessel, wherein the second catheter includes a first end of a second catheter connected to at least one second vessel; a second end of the second catheter to a second buffered flow of the patient; and wherein the flow control apparatus is configured to manipulate a pressure associated with the manipulated flow responsive to manipulating a rate of removal of fluid relative to a rate of introduction of fluid via the first and second catheters.

Still other aspects, embodiments and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment. References to “an embodiment,” “an example,” “some embodiments,” “some examples,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a partially transparent view of one embodiment of a flow control system having a catheter and intracorporeal pump coupled to a flow control apparatus;

FIG. 2 is a partially transparent view of one embodiment of a flow control system having a catheter and intracorporeal pump coupled to a flow control apparatus;

FIG. 3 is a partially transparent view of one embodiment of a flow control system having a catheter and intracorporeal pump coupled to a flow control apparatus;

FIG. 4 is a partially transparent view of one embodiment of a flow control system having a catheter and intracorporeal pump coupled to a flow control apparatus;

FIG. 5 is a partially transparent view of one embodiment of a flow control system having a catheter and flow control apparatus connected to a buffered flow;

FIG. 6 is a partially transparent view of one embodiment of a flow control system having a catheter and flow control apparatus connected to a buffered flow;

FIG. 7 is a partially transparent view of one embodiment of a flow control system having multiple catheters connected to a buffered flow;

FIG. 8 is a partially transparent view of one embodiment of a flow control system having multiple catheters connected to a buffered flow;

FIG. 9 is a partially transparent view of one embodiment of a flow control system;

FIG. 10 is a partially transparent view of one embodiment of a flow control system;

FIG. 11 is a partially transparent perspective view of one embodiment of a flow control system having first and second catheters coupled to a flow control apparatus;

FIG. 12A is a diagram illustrating normal fluid flow through a body part from a fluid inflow vessel to a fluid outflow vessel;

FIG. 12B is a diagram illustrating the fluid flow through the body part of FIG. 12A switched such that fluid flows into the body part from the outflow vessel and fluid exits the body part through the inflow vessel; and

FIG. 13 is a schematic diagram of an exemplary computer system that may be configured to perform processes and functions disclosed herein.

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof as well as additional items.

The present invention generally provides methods and devices for managing fluid flow characteristics, for example, through a body part, such as all or portions of an organ or extremity. In general, fluid vessels with the human body can be managed by a flow control system using catheters, intra-corporeal and/or extra-corporeal pumps. According to one embodiment, the flow control system can deliver intra-corporeal pumps to an affected area of the body (i.e. surrounding an occlusion) and manipulate flow characteristics to resolve the occlusion. According to another embodiment, the flow control system can connect buffered flow from other portions of the human anatomy to provide managed flow to a portion of the body with an obstruction, clot, etc.

In some embodiments, the flow control system enables switching between flow characteristics (e.g., high pressure/high flow, low pressure/high flow, and combinations of high, normal, low pressure, and high, normal, and low flow, and can also include switching directionality of flow, among other options), and optionally can couple buffered blood flow from other portions of the human anatomy to fluid inflow and fluid outflow vessels to deliver managed blood flow to at least a portion of a body part. In some examples, the flow can be switched such that all fluid in at least a portion of the inflow and outflow vessels flows in an opposite direction. In further examples, the flow control system can oscillate between orthodromic flow (i.e., normal direction flow (e.g., artery to vein)) and antidromic flow (i.e., reversed flow (e.g., vein to artery)) to resolve obstructions and/or perfuse oxygen to blocked areas. The flow control system may also be configured to oscillate between high pressure/high flow, low pressure/high flow, combinations of high, normal, low pressure, and high, normal, and low flow, and can include switching directionality of flow, among other options.

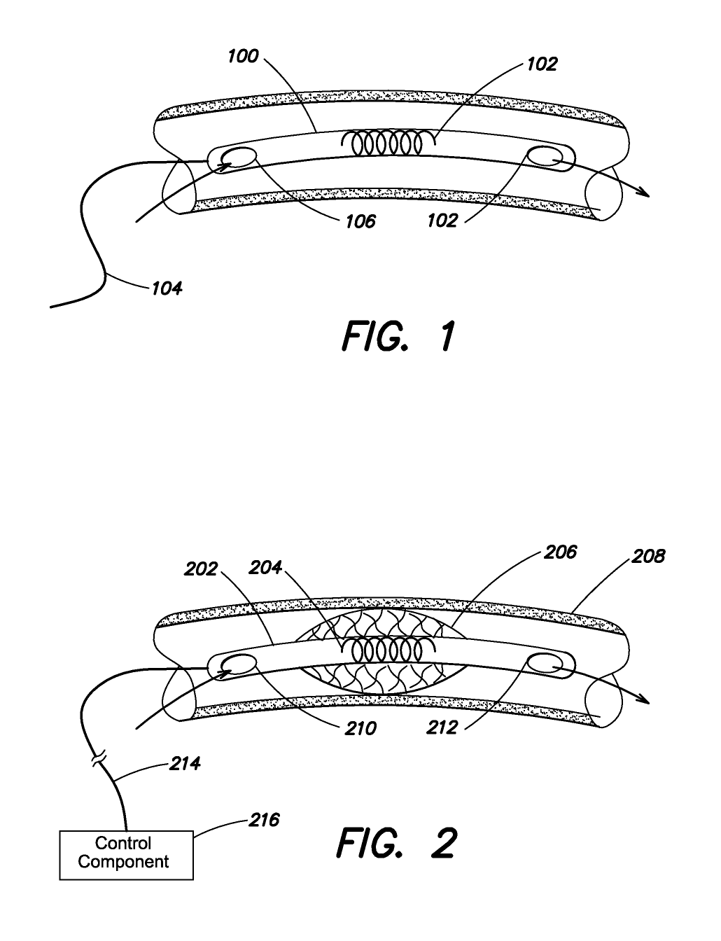

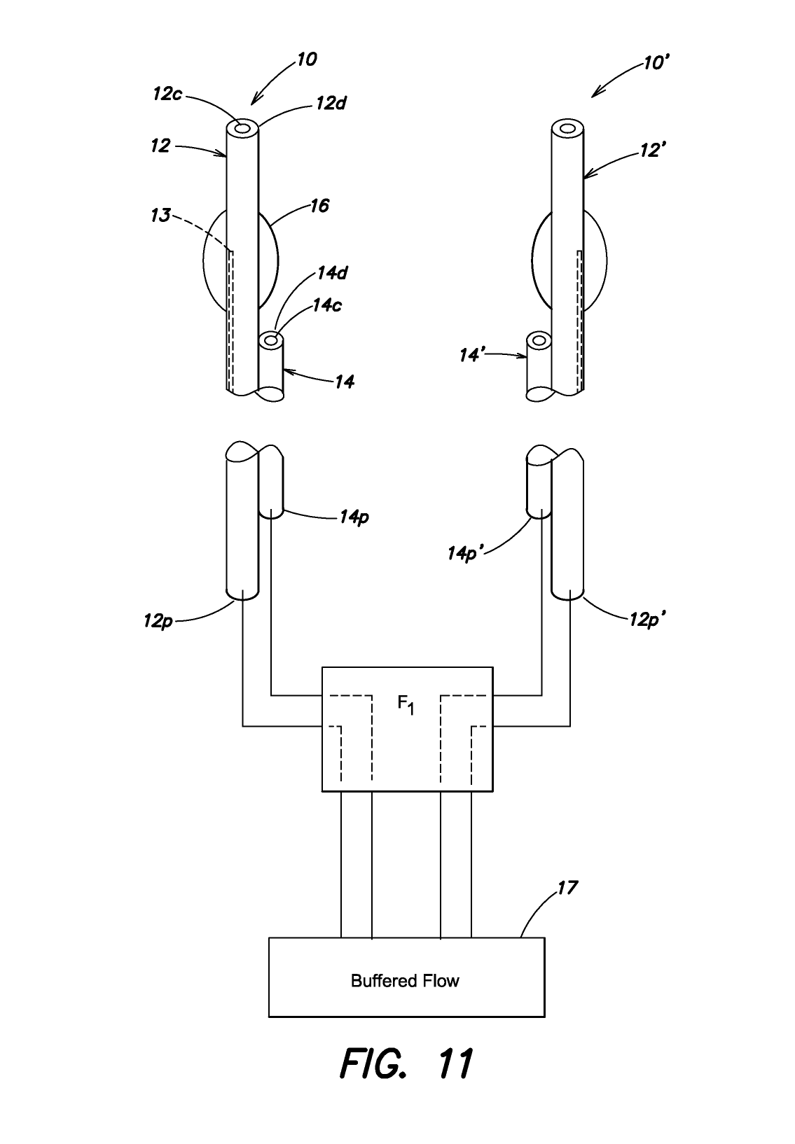

According to one embodiment, the flow control system is configured to form at least one blockage in at least one vessel and to control fluid flow characteristics through the vessel(s). While the flow control system can have virtually any configuration, in one embodiment the system includes one or more balloon catheters and one or more intra-corporeal pumps. A person skilled in the art will appreciate that various other hollow elongate members having various expandable elements formed thereon can be used in place of the balloon catheters discussed herein. FIG. 1 illustrates one exemplary embodiment of a catheter 100 and intra-corporeal pump 102 that can be used to manipulate flow characteristics. According to one embodiment, the catheter and pump can include a control wire 104 configured to operate the pump 102. In one example, the pump 102 can include the known Impella pump commercially available from Abiomed. Although in other examples, difference pumps can be used within catheter 100. In various embodiments, the catheter and pump can be configured to manipulate flow characteristics within a blood vessel by pulling blood into inlet 106 and directing blood through outlet 108 responsive to control signals delivered over the control wire 104. In further examples, the control wire 104 can be omitted and the control signals delivered wirelessly. Not shown is a flow controller configured to deliver control signals to the pump 102. The flow controller can be a specially configured general purpose computer system (e.g., 1100 and/or 1102 as discussed with respect to FIG. 11).

According to some embodiments, the flow control system (e.g., via control signals delivered to the pump) can manipulate blood flow while maintaining orthodromic flow or reversing normal blood flow. In some examples, blood flow manipulation can be accomplished with arterial side manipulation only, venous side manipulation only, and/or combinations of arterial and venous side manipulation. For example, the catheter and pump of FIG. 1 can be introduced in an artery of a patient. A flow control system can be configured to deliver control signals to the pump to alter flow characteristics of the patient's blood. For example, the flow control system can be configured to achieve any one or more of: Hi Flow with NO change in Pressure; Hi Flow with Decreased Pressure; Hi Flow with Increased Pressure; Medium Flow (Baseline Flow or normal flow for patient) with no change in Pressure; Medium Flow (Baseline Flow) with Decreased Pressure; Medium Flow (Baseline Flow) with Increased Pressure; Low Flow with no change in Pressure; Low Flow with Decreased Pressure; and Low Flow with Increased Pressure. According to some embodiments, the flow control system can be configured to provide the preceding options for both orthodromic and antidromic flow scenario. Further, the flow control system can be configured to oscillate between any of the flow options coupled to either flow direction.

Various embodiments of the flow control system can use different implementations of catheters and intra-corporeal pumps. Additionally, the flow control system can employ catheters connected to extra-corporeal pumps. Shown in FIG. 2 is another example catheter 202 and pump 204 that can be used in a flow control system. The catheter 202 includes an expandable portion 206 configured to mate with the walls of a patient's blood vessel 208. The expandable portion 206 mates with the walls of the patient's blood vessel to block blood flow except as provided under the control of the pump 200 through openings 210 and 212. In one example, the expandable portion 206 includes a balloon. Other embodiments can include different structures at 206 to block a blood vessel. In one example, the operation of the pump 204 can be controlled via control signals communicated over control wire 214, which can be operatively connected to a control component 216. Responsive to control signals from the control component 216, the pump 204 can be configured to manipulate the flow characteristics of the patient's blood. In one example, opening 210 can operate as an inlet for blood flow manipulated by pump 204 and direct through opening 212 operating as an outlet for blood flow.

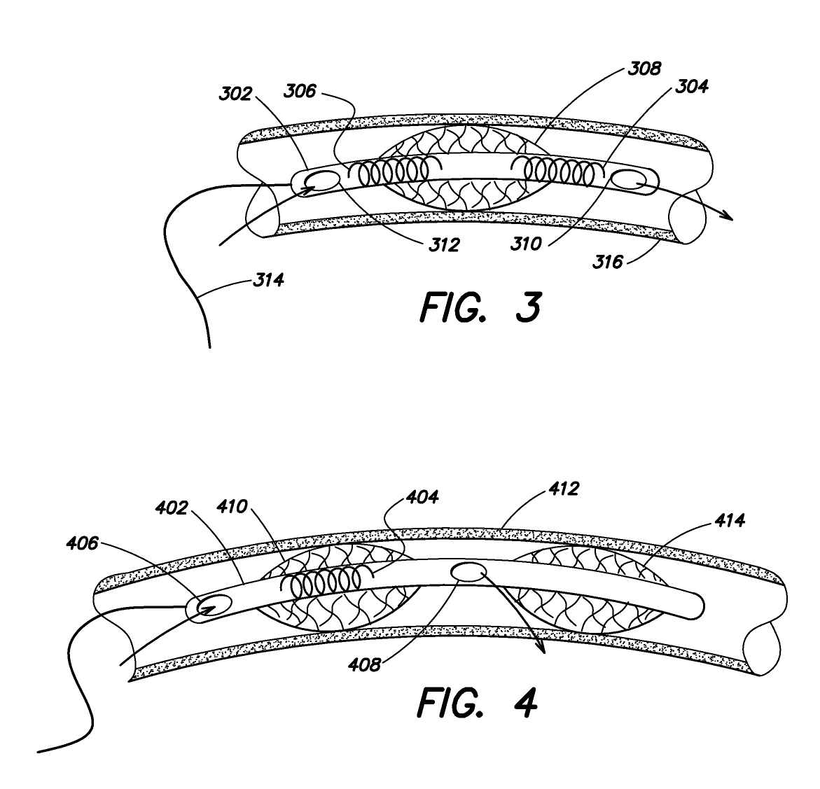

Shown in FIG. 3 is another embodiment of a catheter and pump configuration used in a flow control system. The catheter 302 can include more than one pump (e.g., 304 and 306). According to one embodiment, the catheter 302 includes an expansion member 308 (e.g., a balloon). The expansion member is configured to block blood flow when expanded, forcing any flow in the vessel through openings 310 and 312 in the catheter 302. The flow control system can be configured to block blood flow through a patient's blood vessel, to facilitate control of the blood flow characteristics within the blood vessel. The flow control system can include a control wire connected to any number of pumps in the catheter. In some embodiments, the pumps 304 and 306 can be configured to operate in opposed directions. The pumps 304 and 306 can also be configured to provide continuation operation and pulse or oscillating operation. In some examples, a continuous flow pump can be paired with an oscillating pump oriented in an opposed direction. Control signals received over the control wire 314 can selectively operate the pumps 304 and 306 to manipulate flow characteristics within the blood vessel 316 (e.g., orthodromic flow having: Hi Flow with NO change in Pressure; Hi Flow with Decreased Pressure; Hi Flow with Increased Pressure; Medium Flow (Baseline Flow) with NO change in Pressure; Medium Flow (Baseline Flow) with Decreased Pressure; Medium Flow (Baseline Flow) with Increased Pressure; Low Flow with NO change in Pressure; Low Flow with Decreased Pressure; and Low Flow with Increased Pressure). According to one embodiment, manipulation of flow characteristics can also include reversing the blood flow and reversed flow characteristics (Hi Flow with NO change in Pressure; Hi Flow with Decreased Pressure; Hi Flow with Increased Pressure; Medium Flow (Baseline Flow) with NO change in Pressure; Medium Flow (Baseline Flow) with Decreased Pressure; Medium Flow (Baseline Flow) with Increased Pressure; Low Flow with NO change in Pressure; Low Flow with Decreased Pressure; and Low Flow with Increased Pressure).

Shown in FIG. 4 is another embodiment of a catheter and pump configuration used in a flow control system. The catheter 402 can include one or more pumps (e.g., 404) configured to manipulate flow characteristics of the patient's blood flow. The one or more pumps (e.g., 404) can be configured to control blood flow through openings 406 and 408. The catheter can include a first balloon (e.g., 410) or expansion member configured to seal a blood vessel. The catheter 402 can also include a second balloon (e.g., 414). The second balloon can be constructed an arranged to create a seal in the blood vessel along a flow path provided by the openings 406 and 408. In one embodiment, the second balloon (e.g., 414) can be manipulated to seal the patient's blood vessel to prevent blood flow through openings 406 and 408. In some embodiments, the flow control system can be configured to manipulate expansion and contraction of the second balloon to manipulate the flow characteristics of the patient's blood flow. In some examples, the flow control system can be configured to stop flow using the second balloon, pulse blood flow through vessel 412, provide increase pressure flow, provide decreased pressure flow, etc. According to some embodiments, a control wire can be connected to the catheter 402. The control wire can provide control signals to the pump 404, the first balloon, and/or the second balloon 414. The flow control system is configured to manipulate flow characteristics (e.g., pressure, rate, volume, direction, etc.) via control signals provided to the pump, and/or the balloons. For example, the system is configured to manipulate flow characteristics by opening and closing the second balloon 414. According to some embodiments, opening and closing the second balloon enables the flow control system to oscillate between different flow characteristics, stop flow, reverse flow, and/or manipulate reversed flow characteristics. FIG. 4 illustrates an example of a catheter having multiple balloons. In other embodiments, the balloon elements can be positioned differently, such that the first balloon member is configured to oscillate between open and closed positions to manipulate flow characteristics. In further embodiments, additional balloon elements can be employed.

According to some implementations, the flow control system can be configured with catheters and intra-corporeal pumps constructed and arranged at the distal end of the catheters. In further embodiments, the flow control system can be constructed and arranged with extracorporeal pumps configured to manipulate flow characteristics in conjunction with balloon catheters introduced into blood vessels. The flow control system can also be configured to manipulate intra and extra-corporeal pumps to manipulate flow characteristics (e.g., pressure, rate, volume, direction, etc.).

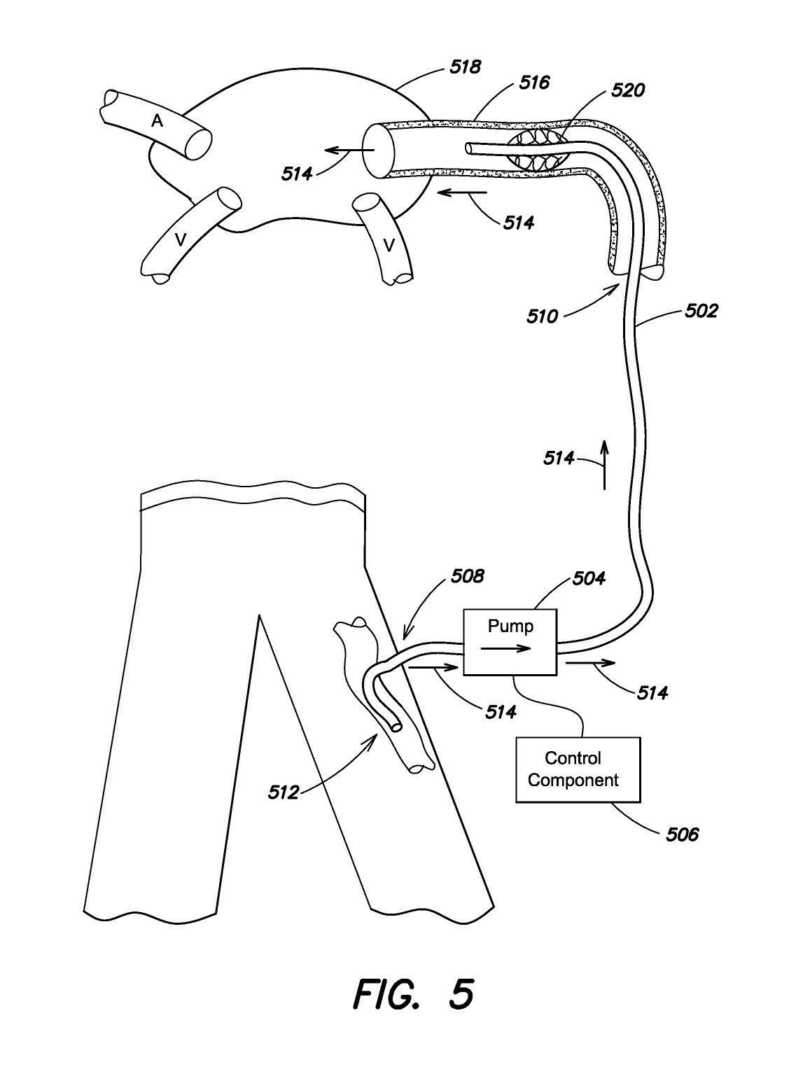

Shown in FIG. 5 is an embodiment of a flow control system 500. The flow control system 500 includes a single lumen catheter 502 connected to an extra-corporeal pump 504 controlled by a control component 506. The single lumen catheter 502 can have two portions 508 and 510. A first portion 508 can be a capture portion where the first portion is configured to capture blood from a buffered flow in the patient. For example, the capture portion of the catheter can be surgically placed within an artery of the patient at 512. In one setting, the patient's femoral artery is selected to provide a buffered blood flow. In one example, capturing blood flow from a patient in a buffer zone minimizes complexity, as the buffered flow has a pressure, rate, and volume normalized to the patient. The directional arrows illustrate blood flow through the first portion 508 to the pump 504. The blood flow continues (514) through the second portion of the catheter 510 (e.g., a delivery portion). The delivery portion of the catheter (e.g., 510) can be placed in an artery 516 of a patient organ 518 or other tissue and/or body part. The flow arrows (514) indicate a blood flow according to an orthodromic flow (i.e. normal flow direction). As discussed above, the control component 506 can provide control signals to the pump 504 to manage flow characteristics of the orthodromic flow (e.g., increase pressure, volume, rate, direction, etc.). In some embodiments, the delivery portion of the catheter can include an expansion portion 520. The expansion portion 520 can be a balloon that expands to form a blockage in the artery 516. In some implementations, the control component 506 can be configured to control the expansion and deflation of the expansion component 520. For example, a control wire (not shown) can carry control signals to the expansion component 520. In other examples, the expansion component can be operated with physical components or other elements as is known. (including e.g., a plunger or other physical mechanism).

As shown in FIG. 5, flow control system 500 includes extra-corporeal pump 504. In further embodiments, the flow control system can include intracorporeal pumps (e.g., disposed at the end of catheter 510). Such embodiments can include the structures discussed above with respect to FIG. 1-4. In some examples, the flow control system can control the operation of extra-corporeal pump 504 and an intracorporeal pump through the control component 506.

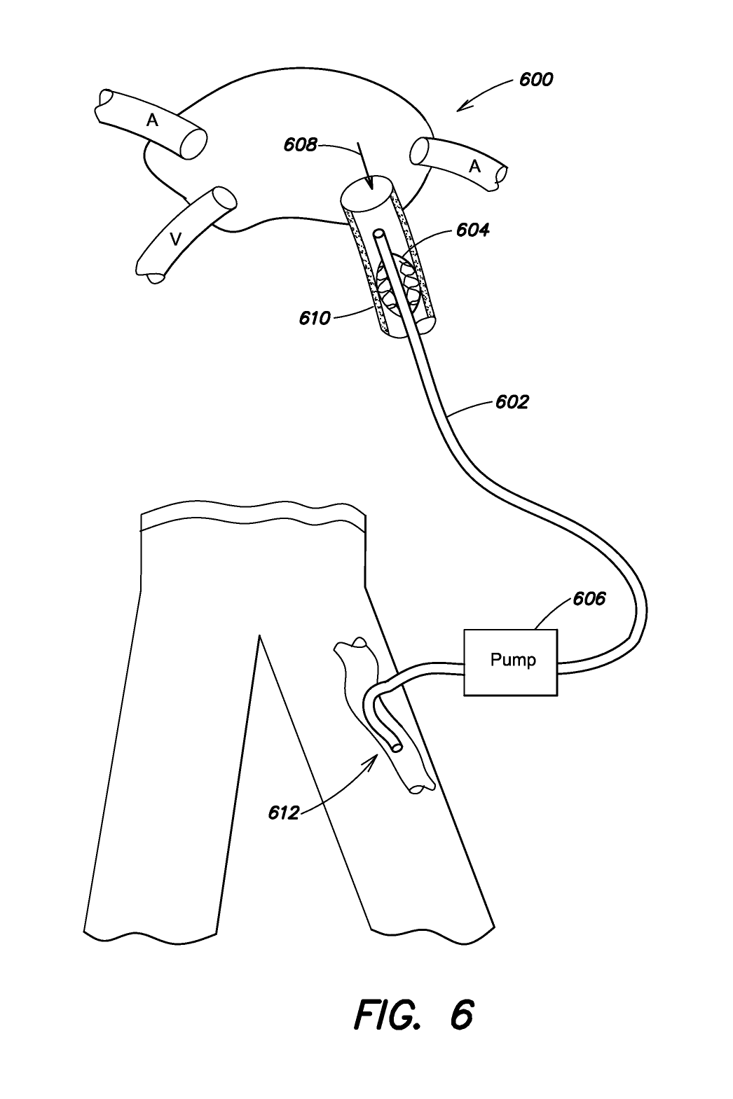

FIG. 6 illustrates another embodiment of a flow control system 600. The flow control system 600 includes a single lumen catheter 602, expansions member 604, and pump 606. As shown, the catheter 602 is placed to capture venous flow from vein 610 and deliver the venous flow to a buffer area at vein 612. In one example, the femoral area of the patient's body provides the buffer area and vein 612 to accept the blood flow captured at 608. According to one embodiment, the pump 606 is configured to manipulate flow characteristics of the blood captured at 608 (e.g., manipulate pressure, rate of flow volume, direction, etc.) and delivered to 612. According to some embodiments, the placement of the catheter (e.g., 602 and 502) is dependent upon where in a patient's body a blockage, clot, or occlusion occurs. In various embodiments, single catheters can be placed proximate to the occlusion. According to some examples, blood flow characteristics can then be manipulated to resolve the occlusion. In some alternatives, the flow control system can be positioned to suffuse oxygenated blood past an occlusion to prevent any damage to tissues, organs, etc. In other embodiment, the flow control system can also include intracorporeal pumps (not shown).

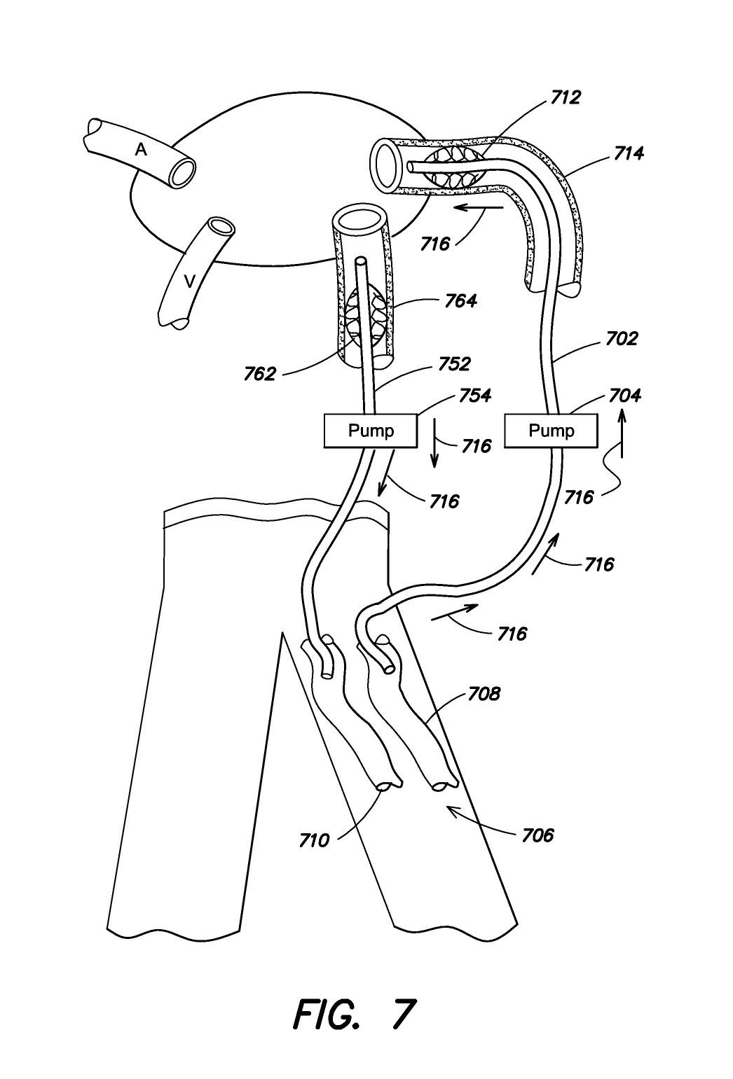

According to one aspect, the flow control system can be used to manipulate flow characteristics on a single side or either side of an occlusion. For example, catheters can be positioned for arterial side manipulation or venous side manipulation. According to other aspects, the flow control system can implement additional flow control manipulations using multiple catheters positioned on both sides of an occlusion. FIG. 7 illustrates another embodiment of a flow control system 700 configured to manipulate flow characteristics by operation on both arterial and venous flow for a patient. According to one embodiment, the flow control system includes two catheters (702 and 752), two extra-corporeal pumps (704 and 754), where the catheters are positioned to capture and return blood to a buffer zone 706 in the patient's anatomy. For example, the buffer zone 706 can be located at the patient's femoral artery 708 and associated vein 710. Each catheter (702 and 752) includes an expansion member (e.g., 712 and 762—which can be a balloon) for providing a blockage in a target blood vessel (e.g., artery 714 and vein 764). The arrows at 716 illustrate a blood flow path, which follows the normal course of blood flow through the patient (i.e., orthodromic flow).

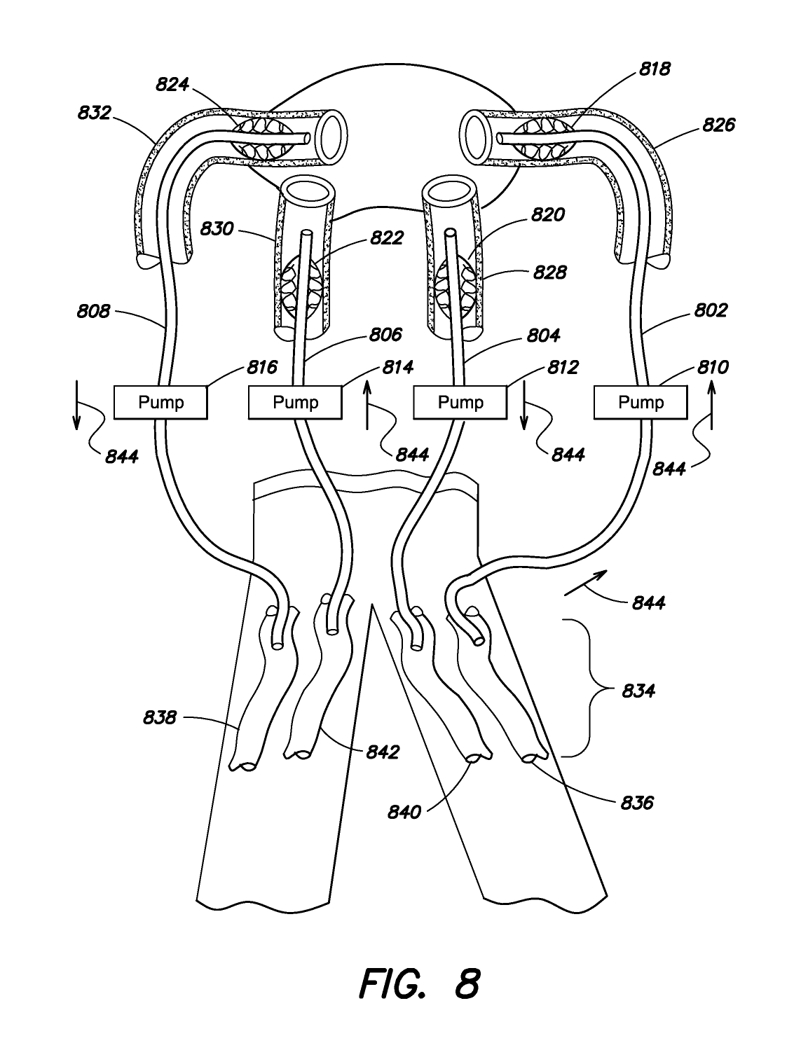

FIG. 8 illustrates another embodiment of a flow control system 800. The flow control system 800 includes four catheters 802, 804, 806, and 808, four extracorporeal pumps 810, 812, 814, and 816. Each catheter can include a respective expansion member (e.g., 818, 820, 822, and 824) configured to provide a blockage in a blood vessel (e.g., artery 826, veins 828 and 830, and artery 832) controlled by the flow control system 800. Each catheter can terminate in a buffer zone 834 (e.g., femoral arteries 836 and 838, and veins 840 and 842) which provides a buffer blood flow to supply extra blood flow (e.g., arteries 836-838 and receive excess capacity (e.g., at veins 840-842). As show in FIG. 8, the flow control system 800 manages orthodromic flow of blood in the patient (e.g., blood flow from artery to vein at flow arrows 844). In other examples, the flow control system 800 can provide control signals to cause the pumps (e.g., any one or more of 810-816) to reverse the blood flow shown (i.e., generate antidromic flow) and/or change the flow characteristics (e.g., flow rate, flow volume, flow pressure, etc.).

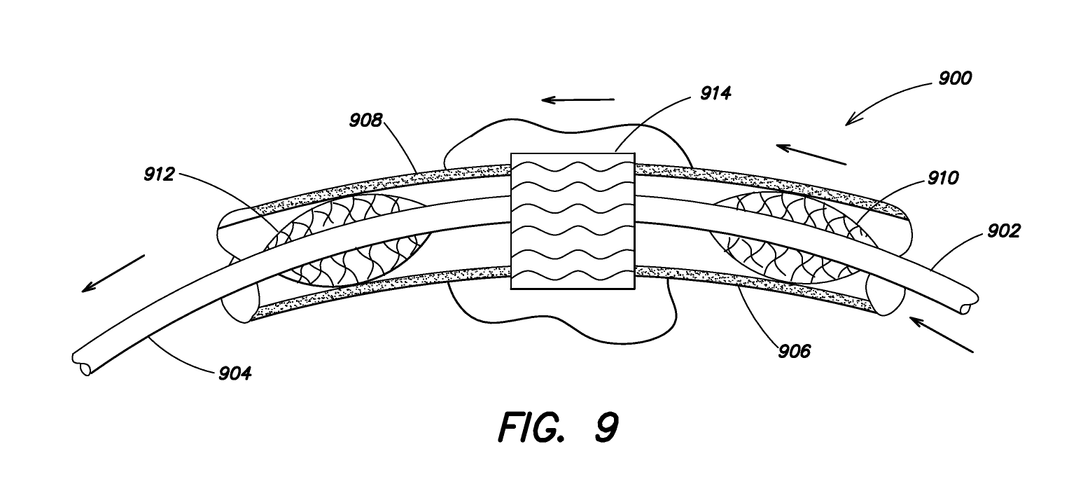

According to another aspect, the effect of arterial and venous control, for example, through a flow control system is illustrated with reference to FIG. 9. Flow control system 900 can include at least a first 902 and second catheter 904. The first and second catheters can be placed in an arterial blood vessel 906 and an associated venous blood vessel 908. Each catheter can include respective expansion members (e.g., 910 and 912) configured to block a respective blood vessel. The arterial vessel 906 delivers oxygenated blood to body part or organ through capillary beds (e.g., shown at 914). Oxygen is delivered and blood flow continues out of the body part or organ through the capillary beds and into vein 908. Once the flow control system has been installed to manage blood flow, the flow control system can manipulate flow characteristics of the blood in the managed region. For example, by manipulating an amount of blood introduced into 902, the flow control system can provide high flow (high flow is defined as a flow that exceeds a baseline (i.e. normal flow) for the patient. If the amount of blood in the artery 902 and corresponding vein 908 is greater than the baseline flow for the patient, the flow control system is providing a high flow state.

According to one embodiment, the flow control system 900 can also manipulate the pressure associated with such high flow. For example, if blood is introduced in 902 at a rate greater than the rate blood is removed in 908, the result is increased pressure over a patient baseline pressure which is referred to as high pressure. In another example, if blood is introduced in 902 at a rate lower than the rate blood is removed from 908, the result is decreased pressure over a patient baseline pressure which is referred to as low pressure. In a further example, if blood is introduced in 902 at a rate equal to the rate blood is removed in 908, the result is normal pressure (i.e., equal or roughly equal to a patient baseline pressure) which is referred to as normal pressure. The flow control system can be configured to provide high pressure, low pressure, and normal pressure in high flow settings, normal flow settings, and low flow settings (where the volume of blood flowing between 902 and 908 is less than a baseline volume).

The embodiments of the flow control system discussed above can be implemented to have different anatomical, hemodynamic, and clinical effects on obstructed vasculature and for example, affected brain tissue, including the penumbra and surrounding zones of the brain.

Embodiments of the flow control system (e.g., 500-900) have been illustrated to show an orthodromic blood flow. In other embodiments, the pumps illustrated are configured to reverse the direction of blood flow, providing antidromic flow, where the flow control system can provide high flow volume, normal flow volume, and low flow volume, couples with pressure control for high pressure, normal pressure, and low pressure, for example, as discussed with respect to FIG. 9. FIGS. 1-9 illustrate and describe example flow control systems and catheters that can be used to manipulate blood flow characteristics. Various configurations of the flow control system and catheters can be used to manipulate blood flow.

According to some embodiments, structural variations in the flow control system and/or catheters used can include the use of one or more catheters constructed of at least one single lumen or one or more catheters constructed of two lumens (including, for example, double lumen catheters). For some embodiments including double lumen catheters, each lumen is constructed to run along a longitudinal axis of the catheter having two ends, a proximal and a distal end. The respective distal ends of the two lumens are located near the distal end of the catheter and are separated by a certain length at the said distal end, providing a spacing between the distal ends of the two lumens. In one embodiment, an expansion member (e.g., a balloon) can be fitted to the distal end of the catheter between the distal openings of each lumen. An extracorporeal pump can be fitted to the proximal end of the catheter. A pump can also be fitted to the distal end of the catheter providing an intracorporeal pump. In some examples, the catheter can be a means to deliver a distally positioned intracorporeal pump, wherein said pump has two openings on its proximal and distal ends on either side of an expansion member (e.g., a balloon). According to one embodiment, the catheter can influence one or more of blood flow characteristics in the vessel where it is deployed. The characteristics include manipulation of flow, pressure, direction of flow and or mixing of two flows inside a target vessel.

According to some embodiments, changes in blood flow characteristics are achieved with single lumen, single opening catheter, double lumen/double distal ends catheters, catheters fitted with two balloons each, one balloon or no balloon at distal end, catheters connected to extracorporeal pumps (unidirectional or bidirectional pumps), catheters carrying terminal intracorporeal pumps with flow assist on one lumen, double lumens/two terminal openings, multi-lumens or no blood lumens. In case of no blood lumen catheters, the flow of blood is through the terminal ends of the distally fitted intracorporeal pump.

According to some implementations, the various embodiment described above can be used to accomplished a variety of flow control options. Based on placement of the components of the flow control system manipulation can be directed to a patient's arterial flow, venous flow and various combination of arterial and venous. In some embodiments, arterial side only manipulation can include using any one or more of: single unilateral catheter in the arterial system (one catheter, one vessel); double unilateral catheters in the Arterial system (two arteries on the same side of the neck); Triple Unilateral Catheters in the Arterial system (three arteries including the Vertebral Artery on the same side of the neck); Single Bilateral Catheters in the Arterial system (one artery on each side of the neck); Multiple bilateral catheters in the Arterial system (two arteries on one side of the neck and one artery on the other side); Multiple bilateral catheters in the Arterial system (three arteries on each side of the neck, including the Vertebral, and one artery on the other side); multiple bilateral catheters in the Arterial system (two arteries on one side of the neck and two arteries on the other side); Multiple bilateral catheters in the Arterial system (three arteries on each side of the neck, including the Vertebral, and two arteries on the other side); and Multiple bilateral catheters in the Arterial System (three arteries on each side of the neck, where the neck positions include vertebral positions, and three arteries on the other side of the neck), among other examples.

In further embodiments, venous side only manipulation can include using any one or more of: single unilateral Catheter in the Venous System (one catheter, one vessel); Double Unilateral Catheters in the Venous System; Triple unilateral catheters (including the Vertebral Vein) Unilateral Catheters in the Venous System; Single Bilateral Catheters in the Venous System (one on each side of the neck); multiple bilateral Catheters in the Venous System (two veins on one side of the neck and one vein on the other side); multiple bilateral Catheters in the Venous System (three veins on each side of the neck, including the Vertebral, and one vein on the other side); multiple Bilateral Catheters in the Venous System (two veins on one side of the neck and two veins on the other side); multiple bilateral Catheters in the Venous System (three veins on each side of the neck, including the Vertebral, and two veins on the other side); and multiple bilateral Catheters in the Venous System (three veins on each side of the neck, including the Vertebral, and three veins on the other side), among other examples.

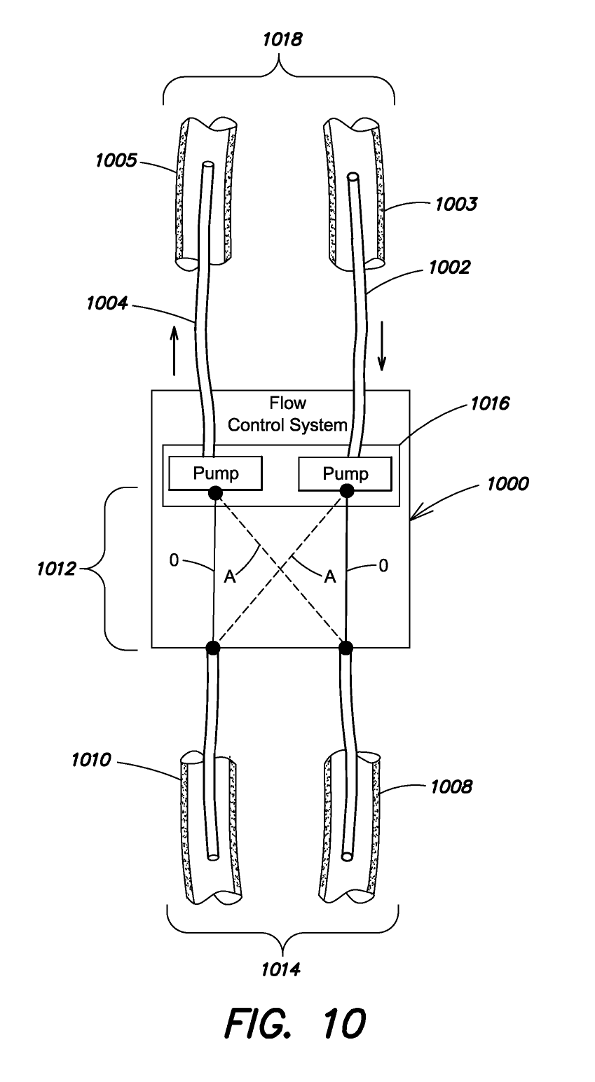

Additional embodiments can combine arterial side manipulations and venous side manipulation. In some embodiments, the flow control system can also include switchable flow paths, such that in an orthodromic flow setting, a first catheter delivers blood to an artery drawn from an artery in the buffer zone of the patient, while a second catheter removes blood from a vein and delivers to a vein in a buffer zone of the patient. The flow control system can be configured to reverse the blood flow and switch the flow paths of the first and second catheter such that in the antidromic setting, the first catheter removes blood from the artery and delivers it a the vein in the buffer zone while the second catheter delivers blood to a vein captured from an artery in the buffer zone. Shown in FIG. 10 is an embodiment of a flow control system 1000. The flow control system includes at least a first catheter 1002, a second catheter 1004. Under orthodromic flow (path O), catheter 1002 delivers blood to artery 1003 along flow captured from artery 1008 (e.g., a femoral artery) in a buffer zone of the patient according to flow path O. Catheter 1004 removes blood from vein 1005 and delivers it to a vein 1010 in the buffer zone of the patient following flow path O. The flow control system can include flow control components (e.g., at 1012) that manage flow paths between the buffer zone 1014 and the target areas 1018. In one example, the flow control components can include one or more pumps 1016 configured to operate on flow paths O and A (e.g., increase flow rate, pressure, etc. along either path). The flow control components can include switching mechanisms that switch between flow paths O and A. For example, the flow control system 1000 can reverse the direction of blood flow (e.g., orthodromic to antidromic flow) and switch the flow paths from O to A in response to reversing an orthodromic flow to an antidromic flow.

According to some embodiments, combined arterial and venous control by a flow control system can include: single unilateral catheter in the arterial and venous system (one artery, one vein, one catheter for each), on one side of the neck; multiple unilateral catheter in the arterial and venous system (two arteries, one vein, one catheter for each), on one side of the neck; multiple unilateral catheter in the arterial and venous system (three arteries, one vein, one catheter for each), on one side of the neck; multiple unilateral catheter in the arterial and venous system (one artery, two veins, one catheter for each), on one side of the neck; multiple unilateral catheter in the arterial and venous system (two arteries, two veins, one catheter for each), on one side of the neck; multiple unilateral catheter in the arterial and venous system (three arteries, two veins, one catheter for each), on one side of the neck; multiple unilateral catheter in the arterial and venous system (one artery, three veins, one catheter for each), on one side of the neck; multiple unilateral catheter in the arterial and venous system (two arteries, three veins, one catheter for each), on one side of the neck; multiple unilateral catheter in the arterial and venous system (three arteries, three veins, one catheter for each), on one side of the neck; multiple bilateral catheter in the arterial and venous system (one artery, one vein, one catheter for each), on both sides of the neck; multiple bilateral catheter in the arterial and venous system (two arteries, one vein, one catheter for each), on both sides of the neck; multiple bilateral catheter in the arterial and venous system (three arteries, one vein, one catheter for each), on both sides of the neck; multiple bilateral catheter in the arterial and venous system (one artery, two veins, one catheter for each), on both sides of the neck; multiple bilateral catheter in the arterial and venous system (two arteries, two veins, one catheter for each), on both sides of the neck; multiple bilateral catheter in the arterial and venous system (three arteries, two veins, one catheter for each), on both sides of the neck; multiple bilateral catheter in the arterial and venous system (one artery, three veins, one catheter for each), on both sides of the neck; multiple bilateral catheter in the arterial and venous system (two arteries, three veins, one catheter for each), on both sides of the neck; and multiple bilateral catheter in the arterial and venous system (three arteries, three veins, one catheter for each), on both sides of the neck, among other examples.

Each of the implementations where catheters are placed arterial side only, venous side only, and in various combinations of arterial and venous placement can be implemented with intracorporeal pumps, extra-corporeal pumps, and combinations of intracorporeal and extracorporeal pumps. Additionally each of the pumps can be configured for unidirectional control (i.e., pump in a single direction) and bi-directional control.

The results of the pump selection are various embodiments of the flow control system having various configurations. The configuration can include, example, (assuming arterial side manipulation) any one or more of: single unilateral catheter in the arterial system (one catheter, one vessel) with one unidirectional extracorporeal pump; single unilateral catheter in the arterial system (one catheter, one vessel) with one bidirectional extracorporeal pump; single unilateral catheter in the arterial system (one catheter, one vessel) with one unidirectional intracorporeal pump; single unilateral catheter in the arterial system (one catheter, one vessel) with one bidirectional intracorporeal pump; double unilateral catheters in the arterial system (two arteries on the same side of the neck) with at least one pump attachment to at least one catheter—the pump is extra or intracorporeal, unidirectional or bidirectional; triple unilateral catheters in the arterial system (three arteries including the Vertebral Artery on the same side of the neck) with at least one pump attachment to at least one catheter—the pump can be extra or intracorporeal, unidirectional or bidirectional; single bilateral catheters in the arterial system (one artery on each side of the neck) with at least one pump attachment to at least one catheter—the pump can be extra or intracorporeal, unidirectional or bidirectional; multiple bilateral catheters in the arterial system (two arteries on one side of the neck and one artery on the other side) with at least one pump attachment to at least one catheter—the pump can be extra or intracorporeal, unidirectional or bidirectional; multiple bilateral catheters in the arterial system (three arteries on each side of the neck, including the Vertebral, and one artery on the other side) with at least one pump attachment to at least one catheter—the pump can be extra or intracorporeal, unidirectional or bidirectional; multiple bilateral catheters in the arterial system (two arteries on one side of the neck and two arteries on the other side) with at least one pump attachment to at least one catheter—the pump can be extra or intracorporeal, unidirectional or bidirectional; multiple bilateral catheters in the arterial system (three arteries on each side of the neck, including the Vertebral, and two arteries on the other side) with at least one pump attachment to at least one catheter—the pump can be extra or intracorporeal, unidirectional or bidirectional; multiple bilateral catheters in the arterial system (three arteries on each side of the neck, including the Vertebral, and three arteries on the other side) with at least one pump attachment to at least one catheter—the pump can be extra or intracorporeal, unidirectional or bidirectional, among other examples.

According to other embodiments, the configurations can include, for example, (assuming venous side manipulation) any one or more of: single unilateral catheter in the venous system (one catheter, one vessel) with one unidirectional extracorporeal pump; single unilateral catheter in the venous system (one catheter, one vessel) with one bidirectional extracorporeal pump; single unilateral catheter in the venous system (one catheter, one vessel) with one unidirectional intracorporeal pump; single unilateral catheter in the venous system (one catheter, one vessel) with one bidirectional intracorporeal pump; double unilateral catheters in the venous system (two veins on the same side of the neck) with at least one pump attachment to at least one catheter—where the pump can be extra or intracorporeal, unidirectional or bidirectional; triple unilateral catheters in the venous system (three veins including the Vertebral Vein on the same side of the neck) with at least one pump attachment to at least one catheter—where the pump can be extra or intracorporeal, unidirectional or bidirectional; single bilateral catheters in the venous system (one vein on each side of the neck) with at least one pump attachment to at least one catheter—where the pump can be extra or intracorporeal, unidirectional or bidirectional; multiple bilateral catheters in the venous system (two veins on one side of the neck and one vein on the other side) with at least one pump attachment to at least one catheter—where the pump can be extra or intracorporeal, unidirectional or bidirectional; multiple bilateral catheters in the venous system (three veins on each side of the neck, including the Vertebral, and one vein on the other side) with at least one pump attachment to at least one catheter—where the pump can be extra or intracorporeal, unidirectional or bidirectional; multiple bilateral catheters in the venous system (two veins on one side of the neck and two veins on the other side) with at least one pump attachment to at least one catheter—where the pump can be extra or intracorporeal, unidirectional or bidirectional; multiple bilateral catheters in the venous system (three veins on each side of the neck, including the Vertebral, and two veins on the other side) with at least one pump attachment to at least one catheter—where the pump can be extra or intracorporeal, unidirectional or bidirectional; multiple bilateral catheters in the venous system (three veins on each side of the neck, including the Vertebral, and three veins on the other side) with at least one pump attachment to at least one catheter—where the pump can be extra or intracorporeal, unidirectional or bidirectional

According to other embodiments, the configurations can include, for example, (assuming arterial and venous side manipulation) any one or more of: single unilateral catheter in the arterial and venous system (one artery, one vein, one catheter for each), on one side of the neck with one unidirectional extracorporeal pump; single unilateral catheter in the arterial and venous system (one artery, one vein, one catheter for each), on one side of the neck with one unidirectional intracorporeal pump; single unilateral catheter in the arterial and venous system (one artery, one vein, one catheter for each), on one side of the neck with one bidirectional extracorporeal pump; single unilateral catheter in the arterial and venous system (one artery, one vein, one catheter for each), on one side of the neck with one bidirectional intracorporeal pump; multiple unilateral catheter in the arterial and venous system (two arteries, one vein, one catheter for each), on one side of the neck with at least one pump attachment to at least one catheter—where the pump can be extra or intracorporeal, unidirectional or bidirectional; multiple unilateral catheter in the arterial and venous system (three arteries, one vein, one catheter for each), on one side of the neck with at least one pump attachment to at least one catheter—where the pump can be extra or intracorporeal, unidirectional or bidirectional; multiple unilateral catheter in the arterial and venous system (one artery, two veins, one catheter for each), on one side of the neck with at least one pump attachment to at least one catheter—where the pump can be extra or intracorporeal, unidirectional or bidirectional; multiple unilateral catheter in the arterial and venous system (two arteries, two veins, one catheter for each), on one side of the neck with at least one pump attachment to at least one catheter—where the pump can be extra or intracorporeal, unidirectional or bidirectional; multiple unilateral catheter in the arterial and venous system (three arteries, two veins, one catheter for each), on one side of the neck with at least one pump attachment to at least one catheter—where the pump can be extra or intracorporeal, unidirectional or bidirectional; multiple unilateral catheter in the arterial and venous system (one artery, three veins, one catheter for each), on one side of the neck with at least one pump attachment to at least one catheter—where the pump can be extra or intracorporeal, unidirectional or bidirectional; multiple unilateral catheter in the arterial and venous system (two arteries, three veins, one catheter for each), on one side of the neck with at least one pump attachment to at least one catheter—where the pump can be extra or intracorporeal, unidirectional or bidirectional; multiple unilateral catheter in the arterial and venous system (three arteries, three veins, one catheter for each), on one side of the neck with at least one pump attachment to at least one catheter—where the pump can be extra or intracorporeal, unidirectional or bidirectional; multiple bilateral catheter in the arterial and venous system (one artery, one vein, one catheter for each), on both sides of the neck with at least one pump attachment to at least one catheter—where the pump can be extra or intracorporeal, unidirectional or bidirectional; multiple bilateral catheter in the arterial and venous system (two arteries, one vein, one catheter for each), on both sides of the neck with at least one pump attachment to at least one catheter—where the pump can be extra or intracorporeal, unidirectional or bidirectional; multiple bilateral catheter in the arterial and venous system (three arteries, one vein, one catheter for each), on both sides of the neck with at least one pump attachment to at least one catheter—where the pump can be extra or intracorporeal, unidirectional or bidirectional; multiple bilateral catheter in the arterial and venous system (one artery, two veins, one catheter for each), on both sides of the neck with at least one pump attachment to at least one catheter—where the pump can be extra or intracorporeal, unidirectional or bidirectional; multiple bilateral catheter in the arterial and venous system (two arteries, two veins, one catheter for each), on both sides of the neck with at least one pump attachment to at least one catheter—where the pump can be extra or intracorporeal, unidirectional or bidirectional; multiple bilateral catheter in the arterial and venous system (three arteries, two veins, one catheter for each), on both sides of the neck with at least one pump attachment to at least one catheter—where the pump can be extra or intracorporeal, unidirectional or bidirectional; multiple bilateral catheter in the arterial and venous system (one artery, three veins, one catheter for each), on both sides of the neck with at least one pump attachment to at least one catheter—where the pump can be extra or intracorporeal, unidirectional or bidirectional; multiple bilateral catheter in the arterial and venous system (two arteries, three veins, one catheter for each), on both sides of the neck with at least one pump attachment to at least one catheter—where the pump can be extra or intracorporeal, unidirectional or bidirectional; multiple bilateral catheter in the arterial and venous system (three arteries, three veins, one catheter for each), on both sides of the neck with at least one pump attachment to at least one catheter—where the pump can be extra or intracorporeal, unidirectional or bidirectional, among other options.

As discussed various embodiments of the flow control system can also be configured to manipulate blood flow characteristics. Various embodiments, discussed in co-pending U.S. Application can be modified to increase management capability. For example, the embodiments, discussed in co-pending application U.S. application Ser. No. 13/550,651 entitled “ARTERIAL-VENOUS SWITCHING,” and filed on Jul. 17, 2012, can be modified to include one or more intracorporeal pumps managed by a flow control system and can also be, in further embodiments, connected to buffered blood flows to permit management of orthodromic and antidromic blood flow for a given organ.

Various aspects and embodiments provide methods and devices for managing fluid flow through a body part as well as managing flow characteristics in normal and switched flow. In the switch flow implementations, the fluid inflow vessel (or at least a portion thereof) becomes a fluid outflow vessel that receives fluid from a body part, and the fluid outflow vessel (or at least a portion thereof) becomes a fluid inflow vessel that delivers fluid to a body part. By switching the direction of fluid flow through at least a portion of a body part, various beneficial results can be achieved. For example, in certain exemplary embodiments, the fluid can be blood, and more preferably oxygenated blood, and the inflow and outflow vessels can be arterial and venous vessels. Switching blood flow through at least a portion of arterial and venous vessels can be accomplished by allowing the healthy venous vessels to act as arterial vessels (or vice versa), thus overcoming problems due to blockage or deterioration. In further embodiments, management of blood flow characteristics can also be used to resolve blockages or deteriorations. For example, oscillations between switched flow and normal flow under control of a flow control system can dislodge a clot. Further oscillations between high pressure, normal pressure, and low pressure in both normal and switched flow directions enables further treatment and can resolve blockages in some examples.

As is known, occlusion of the arterial blood supply to a body part can cause severe damage to vital structures, especially where the occlusion is located in a large vessel, happened acutely or subacutely, and was prolonged. The main reason damage to the organ occurs is because of the lack of oxygen as well as other nutrients delivered by the arterial high pressure blood stream. Thus, in some examples, switching the blood flow allows the blood to reach the organ through the venous pathway, thereby avoiding any further damage to the organ. With the brain, for example, switching oxygenated blood flow into and out of the brain, or at least portions thereof, can prevent further damage from a stroke, since oxygenated blood is now capable of reaching the brain through the fluid outflow vessels (e.g., the venous vessels). Similar benefits can be achieved in various other organs, such as the heart, lungs, liver, etc., as well as in various extremities, such as portions or all of the upper and lower extremities.

While various techniques can be used to manage fluid flow to a body part, in an exemplary embodiment a flow control system is provided and it is configured to form at least one blockage in at least one vessel and to redirect fluid flow through the vessel(s). While the flow control system can have virtually any configuration, in one embodiment the system includes one or more balloon catheters. A person skilled in the art will appreciate that various other hollow elongate members having various expandable elements formed thereon can be used in place of the balloon catheters discussed herein. FIG. 11 illustrates one exemplary embodiment employing a balloon catheter, shown are balloon catheters 10, 10′ coupled to a flow control apparatus F1. Only one of the balloon catheters 10 will be discussed in detail, as the other catheter 10′ can have the same or similar configuration. A person skilled in the art will appreciate that, while two identical balloon catheters are shown, different devices can be used in conjunction with one another.

As shown in FIG. 11, the balloon catheter 10 includes a first tubular member 12 with a lumen 12c extending therethrough and a second tubular member 14 coupled to the first tubular member 12 and having a lumen 14c extending therethrough. One of the tubular members, e.g., the first tubular member 12, can also include an inflation lumen 13, which in the illustrated embodiment is formed in a sidewall thereof, for allowing an inflation fluid, such as air or liquid, to be delivered to an expandable balloon 16 disposed there around for forming a blockage in a pathway. As further shown in FIG. 11, in an exemplary embodiment a distal end 14d of the second tubular member 14 is positioned proximal to the expandable balloon 16 on the first tubular member 12 and the expandable balloon 16 is positioned proximal to a distal end 12d of the first tubular member 12. Such a configuration allows an opening formed in the distal end 12d of the first tubular member 12 to be positioned on one side of the expandable balloon 16 while an opening formed in the distal end 14d of the second tubular member 14 is positioned on an opposite side of the expandable balloon 16.

In use, the openings are thus positioned on opposed sides of a blockage formed in a pathway by the expandable balloon 16. A proximal end 12p, 14p of each tubular member 12, 14 can be coupled to the flow control apparatus F1, as will be discussed below. In some embodiments, the particular configuration of the catheter and the lumens extending therethrough can vary. For example, while the illustrated catheter is shown having two tubular members coupled to one another, in other embodiments a single tubular member can be used with multiple lumens extending therethrough. Accordingly, the term tubular member is intended to include both separate and distinct tubular members that are coupled to one another, and separate and distinct lumens formed through a single tubular member, i.e., a multi-lumen catheter.

The flow control apparatus F1, which is generically represented by a box, can be configured to direct fluid flow between the proximal ends of any number of balloon catheters used with the system and a buffered flow at 17 of a given patient. The buffered flow can be obtained from an artery or vein at another location of the patient's body. For example, femoral blood flow can be connected to the flow control apparatus for delivery or removal through 12c and/or 14c depending on whether the system is configured to provide orthodromic flow or antidromic flow.

In the illustrated embodiment, the flow control apparatus F1 can direct fluid from the proximal ends 12p, 14p of the first and second tubular members 12, 14 of the first balloon catheter 10 to the buffered flow of the patient (e.g., an artery and a vein, two arteries, two veins, etc.). The flow control apparatus F1 can have virtually any configuration for directing fluid flow. For example, F1 can including a housing and one or more pumps with pathways extending there through for switching between buffered flow connections. The pathways can allow the flow control system to switch flow directionality (e.g., between orthodromic and antidromic) while still delivering oxygenated blood to a buffer artery and un-oxygenated blood to a buffered vein (e.g., contained in buffer zone 17) as appropriate. In other embodiments, the flow control system is permitted to mix blood flows and the path switching can be omitted.

In some embodiments, the flow control apparatus F1 can include a control mechanism for allowing a user or executing logic to selectively control the couplings between the proximal ends of the tubular members (e.g., 12p and 12p′) and buffered flow. Such a configuration could include, for example, one or more dials and/or valves that direct fluid flow through the apparatus thus allowing user control over the direction of fluid flow. The one or more dials and/or valves can be responsive to control signals delivered from a computer system (e.g., a flow control component). The fluid control system can also include other features, such as a pump mechanism disposed within or coupled to the flow control apparatus F1 for controlling a rate of fluid flow through the apparatus. In some examples, F1 can also include a recirculating pathway and/or pump for recirculating fluid through the apparatus, and/or one or more drug delivery mechanisms for allowing various drugs to be injected into the fluid and delivered to a desired location.

The flow control system could also include features that would allow various other therapies to be delivered and/or performed, such as dialysis, etc. The flow control system can also optionally control various parameters in addition to flow rate, pressure, and/or chemical or pharmacological composition, such as pulsation, resonance, temperature, and addition or subtraction of components such as blood cells, electrolytes, direct of flow, etc. Various embodiments of the flow control apparatus 10 can have virtually any configuration, and the particular configuration can vary based on the intended use. Moreover, the flow control apparatus 10 need not be a separate housing that is coupled to the balloon catheters 10, 10′, but rather the proximal ends 12p, 14p, 12p′, 14p′ of the balloon catheters 10, 10′ can be configured to directly couple to separate pumps and/or controllers, which are then connected to a buffered flow. The fluid flowing through the system can also be separated or it can be mixed within the system. The system can also be configured to be fully implantable, or various components can remain external to the system. The particular configuration can vary based on whether the system is intended for acute, semi-acute, or long-term/permanent use. While not shown, the system can include other features such as one or more pressure sensors for sensing the fluid pressure within the system and/or expandable balloon.

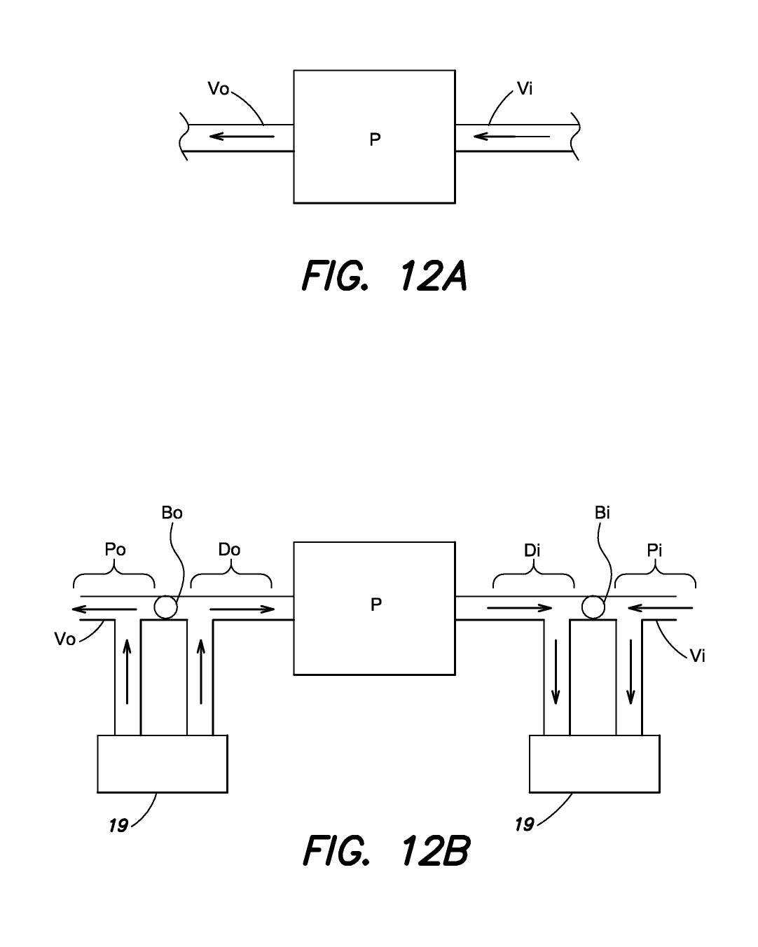

In use, the flow control system can allow fluid flow through at least a portion of a body part to be totally switched. Various embodiments are configured to manage flow to a body part where the body part can include any body part that receives fluid flow therethrough, such as all or certain portions of an organ, vessel, and/or extremity. For example, the body part can be all or certain portions of the brain, heart, lungs, liver, kidney, limb such as an arm or leg, portions of vessels, eye, intestine, adrenal gland, or other regions of the body. The fluid can be any bodily fluid, but in certain exemplary embodiments the fluid is blood, and more preferably oxygenated blood. The term “inflow” vessel is intended to refer to those vessels that naturally deliver fluid to a body part and the term “outflow” vessel is intended to refer to those vessels that naturally receive fluid from a body part. Typically, all body parts have fluid flowing therethrough which is delivered to the body part by an artery and which exits the body part through a vein. Thus, in certain exemplary embodiments, fluid flow is switched by switching the arterial and venous supplies to a body part. The present invention allows the fluid flow through all or a portion of a body part to be managed and/or switched such that the inflow vessel acts as an outflow vessel and the outflow vessel acts as an inflow vessel. FIGS. 12A-B illustrate various exemplary configurations for managing flow when the flow direction is switched.

FIGS. 12A-2B illustrate one exemplary configuration for managing fluid flow when the flow direction has been switched. FIG. 12A illustrates normal fluid flow, showing a fluid inflow vessel Vi for delivering fluid to a body part P, generically represented as a box, and a fluid outflow vessel Vo for receiving fluid flow from the body part P. During normal fluid flow the fluid flows into the body part P from the inflow vessel Vi, and fluid flows away from the body part P through the outflow vessel Vo, as indicated by the arrows in FIG. 12A. With a typical body part, such as the brain, heart, or a limb, blood is delivered to the body part through an arterial vessel and blood flows away from the body part through a venous vessel. In order to switch fluid flow going into and out of the body part, the fluid can be redirected. In particular, as shown in FIG. 12B, a blockage Bi, Bo can be formed in each of the fluid inflow and fluid outflow vessels Vi, Vo to fully block fluid flow therethrough. As indicated above, an expandable balloon on a balloon catheter can be used to form each blockage Bi, Bo, however various other techniques for forming the blockages can also be used. During normal fluid flow in the inflow vessel Vi, fluid will flow toward the blockage Bi on the proximal side Pi of blockage Bi and away from the blockage Bi on the distal side Di of the blockage Bi. As used herein, the term proximal will refer to the side of the blockage that is blocked from communication with the body part P, and the term distal will refer to the side of the blockage that is in fluid communication with the body part P. The terms proximal and distal are not intended to refer to particular anatomical locations, and thus in some cases the terms proximal and distal will refer to a portion of a vessel in the body that is not necessarily anatomically located proximal or distal to the body part. During normal fluid flow in the outflow vessel Vo, fluid will flow away from the blockage Bo on the proximal side Po of blockage Bo (i.e., the side blocked from communication with the body part P) and toward the blockage Bo on the distal side Do of the blockage Bo (i.e., the side in fluid communication with the body part P). Once the blockages Bo, Bi are formed, the fluid flow can be fully managed and/or switched, at least for a portion of the inflow and outflow vessels Vi, Vo, e.g., the portion located on the distal side of each blockage Bi, Bo, for example, to allow fluid to flow into the body part P from the outflow vessel Vo and to flow out of the body part P through the inflow vessel Vi. The pressure of the flow, volume of flow, etc., can be managed by the system.

Additionally, the control can be achieved without affecting the direction of fluid flow on the proximal side Pi, Po of each blockage Bi, Bo, thus allowing fluid flow through other parts of the body to remain unaffected. In order to achieve such switching, as shown in FIG. 12B fluid in the proximal side Pi of the blockage Bi in the inflow vessel Vi can be redirected and delivered to a buffered flow (e.g., an artery), and fluid in the distal side Di of the inflow vessel Vi can be redirected and delivered to a buffered flow 19 (e.g., a vein).

While FIG. 12B generally illustrates two pathways for redirecting and delivering the fluid on each side of the organ, as indicated above the pathways can be in the form of one or more lumens formed in a balloon catheter. For example, the expandable balloon 16 on the first catheter 10 of FIG. 11 can be used to form the blockage Bi in the inflow vessel Vi such that the opening in the distal end 12d of the first tubular member 12 is positioned in fluid communication with the body part B (i.e., on the distal side Di of the blockage), and the opening in the distal end 14d of the second tubular member 14 is positioned on an opposite side of the blockage Bi (i.e., on the proximal side Pi of the blockage) and is thus blocked from fluid communication with the body part B. Fluid on the proximal side Pi of the blockage Bi in the inflow vessel will thus flow into a buffered flow 19 and fluid on the distal side Di of the blockage Bi in the inflow vessel will also flow in a buffered flow 19. In some embodiments, each lumen is connected to a respective vein and artery of the buffered flow. A second catheter can likewise be implanted in the fluid outflow vessel Vo and connected to a buffered flow 19. In further embodiments, each lumen of the first and second catheter is connected to a buffered flow through at a respective vein or artery through a flow control apparatus. Thus, while FIG. 12B illustrates pathways extending directly from the distal side Di, Do of the inflow and outflow vessels Vi, Vo, a person skilled in the art will appreciate that the pathways are merely representative, and that, when a balloon catheter is implanted the lumens will form the pathways.

As a result of the fluid being redirected, fluid flowing in its normal direction toward the blockage Bi on the proximal side Pi of the blockage Bi in the inflow vessel Vi will be delivered to the buffered flow 19, and fluid from the buffered flow will be delivered to the distal side Do of the blockage Bo in the outflow vessel Vo, and will thus flow into the body part P through the outflow vessel Vo, thus switching the direction of fluid flow in the body part P. The fluid will pass through the body part P and will exit the body part P and flow into the inflow vessel Vi, thus switching the direction of fluid flow in the distal side Di of the inflow vessel Vi. The fluid flowing from the body part P into the distal side Di of the inflow vessel Vi will be delivered to the buffered flow 19, for example, into a vein. Buffered flow will also be delivered from the buffered flow 19 to the outflow vessel Vo, where it will flow away from the blockage Bo in its normal direction.

In other implementations, the dual lumen catheters shown in FIG. 11 can be used to mange flow in an orthodromic direction, where other flow characteristics are managed, for example, to resolve obstructions. A person skilled in the art will appreciate that flow management, including switching, can be performed to treat numerous conditions. For example, in other embodiments embolic ischemia secondary to occlusion of any limb arterial supply can be treated by arterial-venous switching to save the affected limb from gangrene. The treatment can also be used in cases of chronic vasculopathy secondary to diseases like diabetes, which mainly affects the arterial side and partially spares the venous side. Arterial-venous switching at the level of the femoral artery and the iliac artery can also be used to treat primary vascular diseases like Burger's disease. Such switching treatment might also prove effective in decreasing the reaction in small or medium sized vessels in the case of connection tissue diseases or vasculitidis that mainly affects the arterial side. Other applications include treatment of internal bleeding or uterine bleeding. In the case of severe acute bleeding that can be life threatening, an immediate arterial-venous switch of the main blood vessels supplying the specific anatomic location might be curative until permanent therapy is implemented. Sometimes the source of bleeding is either obscure or diffuse over a large raw area that can be difficult to control.

In cases of uterine bleeding, sometimes hysterectomy is the only solution to prevent fatal bleeding. Arterial-venous switching in this case can be life saving while sparing the uterus until the wound is healed after a decrease of the blood pressure resulting from the switching. Arterial-venous switching may also provide new solutions to reperfusion of coronary vessels, by switching from atherosclerosis coronary arteries to partially spared coronary veins. Esophageal or stomach bleeding could also be treated by switching portal veins circulation partially to hepatic arterial circulation.

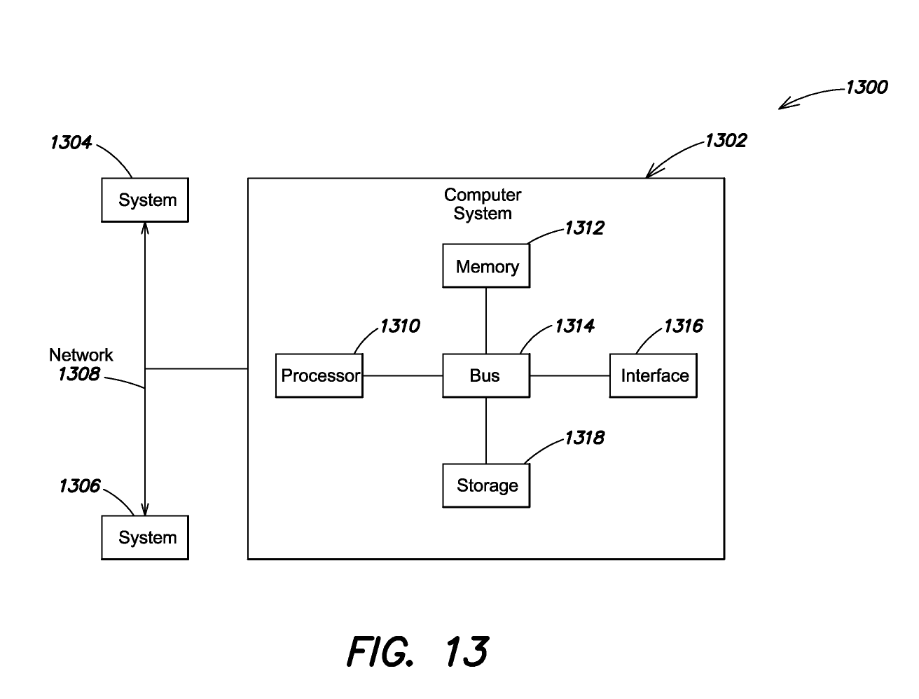

Shown in FIG. 13 is an example computer system on which various flow control procedures can be programmed and/or executed. In some embodiments, a flow control system can include a general purpose computer system (e.g., 1300 and/or 1302) which is specially configured to operate pumps for managing flow characteristics, flow direction, flow rate, flow volume, flow pressure, oscillation between flow states, execute pre-programmed sequences of flow states, etc. In further embodiments, a flow controller can also include a general purpose computer system (e.g., 1300 and/or 1302). In some implementations, the programming associated with the flow control system can be executed and result in synchronized control of multiple pumps, individual pumps and/or provide for independent control of multiple pumps and resulting blood flow characteristics. Further, functions that can be executed can include expansion and contraction of expansion members (e.g., inflation/deflation of balloons), among other options. In some embodiments, expansion and contraction of the expansion member can be used to manage blood flow characteristics.

Various aspects and functions described herein may be implemented as specialized hardware or software components executing in one or more computer systems. There are many examples of computer systems that are currently in use. These examples include, among others, network appliances, personal computers, workstations, mainframes, networked clients, servers, media servers, application servers, database servers and web servers. Other examples of computer systems may include mobile computing devices, such as cellular phones and personal digital assistants, and network equipment, such as load balancers, routers and switches. Further, aspects may be located on a single computer system or may be distributed among a plurality of computer systems connected to one or more communications networks.

For example, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Consequently, examples are not limited to executing on any particular system or group of systems. Further, aspects and functions may be implemented in software, hardware or firmware, or any combination thereof. Thus, aspects and functions may be implemented within methods, acts, systems, system elements and components using a variety of hardware and software configurations, and examples are not limited to any particular distributed architecture, network, or communication protocol.

Referring to FIG. 13, there is illustrated a block diagram of a special purpose distributed computer system 1300, in which various aspects and functions are practiced. As shown, the distributed computer system 1300 includes one more computer systems that exchange information and can perform a variety of functions and in particular: sensing blood flow in a particular region, or lack thereof, responsive to detecting reduced or lack of blood flow manipulating at least one of rate, volume, pressure, and direction of flow within the at least one vessel with an intracorporeal pump; sensing blood flow and varying directionality of blood flow responsive to detecting reduced or lack of blood flow; among other functions. More specifically, the distributed computer system 1300 includes special purpose computer systems 1302, 1304 and 1306. As shown, the computer systems 1302, 1304 and 1306 are interconnected by, and may exchange data through, a communication network 1308.

In some embodiments, the network 1308 may include any communication network through which computer systems may exchange data. To exchange data using the network 1308, the computer systems 1302, 1304 and 1306 and the network 1308 may use various methods, protocols and standards, including, among others, Fibre Channel, Token Ring, Ethernet, Wireless Ethernet, Bluetooth, IP, IPV6, TCP/IP, UDP, DTN, HTTP, FTP, SNMP, SMS, MMS, SS7, JSON, SOAP, CORBA, REST and Web Services. To ensure data transfer is secure, the computer systems 1302, 1304 and 1306 may transmit data via the network 1308 using a variety of security measures including, for example, TLS, SSL or VPN. While the distributed computer system 1300 illustrates three networked computer systems, the distributed computer system 1300 is not so limited and may include any number of computer systems and computing devices, networked using any medium and communication protocol.

As illustrated in FIG. 13, the computer system 1302 includes a processor 1310, a memory 1312, a bus 1314, an interface 1316 and data storage 1318. To implement at least some of the aspects, functions and processes disclosed herein, the processor 1310 performs a series of instructions that result in manipulated data. The processor 1310 may be any type of processor, multiprocessor or controller. Some exemplary processors include commercially available processors such as an Intel Xeon, Itanium, Core, Celeron, or Pentium processor, an AMD Opteron processor, a Sun UltraSPARC or IBM Power5+ processor and an IBM mainframe chip. The processor 1310 is connected to other system components, including one or more memory devices 1312, by the bus 1314.

The memory 1312 stores programs and data during operation of the computer system 1302. Thus, the memory 1312 may be a relatively high performance, volatile, random access memory such as a dynamic random access memory (DRAM) or static memory (SRAM).

However, the memory 1312 may include any device for storing data, such as a disk drive or other non-volatile storage device. Various examples may organize the memory 1312 into particularized and, in some cases, unique structures to perform the functions disclosed herein. These data structures may be sized and organized to store values for particular data and types of data.

Components of the computer system 1302 are coupled by an interconnection element such as the bus 1314. The bus 1314 may include one or more physical busses, for example, busses between components that are integrated within a same machine, but may include any communication coupling between system elements including specialized or standard computing bus technologies such as IDE, SCSI, PCI and InfiniBand. The bus 1314 enables communications, such as data and instructions, to be exchanged between system components of the computer system 1302.

The computer system 1302 also includes one or more interface devices 1316 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 1302 to exchange information and to communicate with external entities, such as users and other systems.

The data storage 1318 includes a computer readable and writeable nonvolatile, or non-transitory, data storage medium in which instructions are stored that define a program or other object that is executed by the processor 1310. The data storage 1318 also may include information that is recorded, on or in, the medium, and that is processed by the processor 1310 during execution of the program. More specifically, the information may be stored in one or more data structures specifically configured to conserve storage space or increase data exchange performance.

The instructions stored in the data storage may be persistently stored as encoded signals, and the instructions may cause the processor 1310 to perform any of the functions described herein. The medium may be, for example, optical disk, magnetic disk or flash memory, among other options. In operation, the processor 1310 or some other controller causes data to be read from the nonvolatile recording medium into another memory, such as the memory 1312, that allows for faster access to the information by the processor 1310 than does the storage medium included in the data storage 1318. The memory may be located in the data storage 1318 or in the memory 1312, however, the processor 1310 manipulates the data within the memory, and then copies the data to the storage medium associated with the data storage 1318 after processing is completed. A variety of components may manage data movement between the storage medium and other memory elements and examples are not limited to particular data management components. Further, examples are not limited to a particular memory system or data storage system.

Although the computer system 1302 is shown by way of example as one type of computer system upon which various aspects and functions may be practiced, aspects and functions are not limited to being implemented on the computer system 1302 as shown in FIG. 13. Various aspects and functions may be practiced on one or more special purpose computers having a different architectures or components than that shown in FIG. 13. For instance, the computer system 1302 may include specially programmed, special-purpose hardware, such as an application-specific integrated circuit (ASIC) tailored to perform a particular operation disclosed herein. While another example may perform the same function using a grid of several special purpose computing devices running MAC OS System X with Motorola PowerPC processors and several specialized computing devices running proprietary hardware and operating systems.

The computer system 1302 may be a computer system including an operating system that manages at least a portion of the hardware elements included in the computer system 1302. In some examples, a processor or controller, such as the processor 1310, executes an operating system. Examples of a particular operating system that may be executed include a Windows-based operating system, such as, Windows NT, Windows 2000 (Windows ME), Windows XP, Windows Vista or Windows 7 or 8 operating systems, available from the Microsoft Corporation, a MAC OS System X operating system available from Apple Computer, one of many Linux-based operating system distributions, for example, the Enterprise Linux operating system available from Red Hat Inc., a Solaris operating system available from Sun Microsystems, or a UNIX operating systems available from various sources. Many other operating systems may be used, and examples are not limited to any particular operating system.

The processor 1310 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, C# (C-Sharp), Objective C, or Javascript. Other object-oriented programming languages may also be used. Alternatively, functional, scripting, or logical programming languages may be used.

Additionally, various aspects and functions may be implemented in a non-programmed environment, for example, documents created in HTML, XML or other format that, when viewed in a window of a browser program, can render aspects of a graphical-user interface or perform other functions.

Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements, e.g. specialized hardware, executable code, data structures or objects, that are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for managing blood flow in a body part, comprising:
coupling a flow control system to the body part, the flow control system configured to provide at least one flow path;
managing blood flow characteristics in at least one vessel connected to the body part along the at least one flow path;
wherein coupling the flow control system to the body part includes placing a catheter and an intracorporeal pump within the catheter into the at least one vessel; and
manipulating at least one of rate, volume, pressure, and direction of flow within the at least one vessel with the intracorporeal pump.

2. The method of claim 1, wherein the catheter and pump is placed entirely within the vessel.

3. The method of claim 1, further comprising expanding an expansion member on the distal end of the catheter to block unmanaged blood flow in the at least one vessel.

4. The method of claim 3, further comprising managing blood flow from an inlet positioned on one side of the expansion member to an outlet positioned on an opposite side of the expansion member.

5. The method of claim 4, wherein managing the blood flow includes operating the intracorporeal pump to affect at least one of rate, volume, pressure, and direction of blow flow.

6. The method of claim 5, wherein the method further comprises switching between flow states, wherein the switching between flow states includes switching between at least one of high flow, normal flow, and low flow coupled with at least one of high pressure, normal pressure, and low pressure.

7. The method of claim 6, wherein the flow states include flow direction.

8. The method of to claim 1, further comprising:
connecting a distal end of the catheter to a buffer zone having a normal fluid flow portion; and
capturing a fluid volume from the buffer zone for delivery to the body part along the at least one flow path.

9. The method of to claim 8, further comprising:
connecting the distal end of the catheter to an artery in the buffer zone; and
capturing a fluid volume from the artery for delivery to the body part along the at least one flow path.

10. The method of to claim 1, further comprising:
connecting a distal end of the catheter to a buffer zone having a normal fluid flow portion; and
delivering a fluid volume from the buffer zone for delivery to the body part along the at least one flow path.

11. The method of to claim 10, further comprising
connecting the distal end of the catheter to a vein in the buffer zone; and
delivering a fluid volume from the body part to the vein for delivery along the at least one flow path.

12. The method of to claim 1, wherein coupling the flow control system to the body part includes placing at least a second catheter and second intracorporeal pump into another blood vessel coupled to the at least one blood vessel; and
wherein manipulating the at least one of rate, volume, pressure, and direction of flow includes manipulating flow characteristics within the at least one vessel and the another blood vessel with the intracorporeal pump and the second intracorporeal pump.

13. A system for managing blood flow in a body part, comprising:
a flow control apparatus configured to:
provide at least one flow path to the body part;
manage blood flow characteristics in at least one vessel connected to the body part along the at least one flow path;
a catheter and an intracorporeal pump within the catheter configured to:
manipulate at least one of rate, volume, pressure, and direction of flow within the at least one vessel with the intracorporeal pump.

14. The system of claim 13, wherein the catheter and intracorporeal pump are configured to fit entirely within the at least one vessel.

15. The system of claim 13, further comprising an expansion member on the distal end of the catheter configured to block unmanaged blood flow in the at least one vessel.

16. The system of claim 15, wherein the catheter further comprises an inlet positioned on one side of the expansion member connected to an outlet positioned on an opposite side of the expansion member.

17. The system of claim 16, wherein the catheters is configured to manage blood flow between the inlet and outlet.

18. The system of claim 17, wherein managing the blood flow includes operating the intracorporeal pump to affect at least one of rate, volume, pressure, and direction of blow flow.

19. The system of claim 13, wherein managing the blood flow comprises switching between flow states, wherein the switching between flow states includes switching between at least one of high flow, normal flow, and low flow coupled with at least one of high pressure, normal pressure, and low pressure.

20. The system of claim 19, wherein the flow states include flow direction.

21-28. (canceled)
